(19) United States
(12) Patent Application Publication
(10) Pub. No.: US 2011/0250254 A1
(43) Pub. Date: Oct. 13, 2011

(54) ANTIMICROBIAL COMPOSITIONS AND USES

(76) Inventors: Anne Aamdal Scheie, Amdalsverk (NO); Tore Benneche, Oslo (NO); Jessica Lonn-Stensrud, Lysaker (NO); Jan Skramstad, Rykkin (NO)

(21) Appl. No.: 13/122,994

(22) PCT Filed: Oct. 9, 2009
(86) PCT No.: PCT/EP2009/063210

§ 371 Date: Jun. 17, 2011

(30) Foreign Application Priority Data
Oct. 9, 2008 (GB) 0818547.2

(51) Int. Cl. A61K 31/381; A61Q 11/00; A61F 2/00; A01N 25/00; A61F 13/00; A01N 25/34; C07D 333/32; A01N 43/10; C07D 409/06; C07D 409/14; A61Q 15/00; A61P 31/00; A01P 1/00; A01P 3/00; C08F 228/06; A61K 8/49

(52) U.S. Cl. 424/405; 424/65; 424/49; 424/408; 549/62; 514/445; 549/66; 549/64; 549/59; 514/444; 526/256

(57) ABSTRACT

An agent comprising the compound according to general formula (I) wherein R₁, R₂, R₃ and R₄ are each independently H or a substituent, and wherein at least one of R₁, R₃ and R₄ is halogen, cyano, cyanate, thiocyanate or C₁-C₆ haloalkyl.

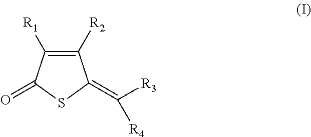

The present invention relates to chemical compounds and polymers incorporating such chemical compounds for use as antimicrobial agents, more particularly for blocking or interfering with quorum-sensing microbial communication and/or preventing or inhibiting biofilm formation.

### BACKGROUND OF THE INVENTION

Microbes, and in particular bacteria, are known to form biofilms under conditions where there is a combination of bacteria, moisture, nutrients and a suitable surface. Biofilm formation and biofouling create problems and economic losses in domestic, industrial and health fields. Various industrial processes and installations may be affected, such as submarine installations and shipping, various engineering industries, oil processing and manufacturing, the food and beverage industry, the pharmaceutical industry, water systems, cooling towers, heat exchangers, chain lubrication systems and the like. Biofilms also cause problems in relation to medical devices and implants and cause various human and animal infections. It is generally necessary to use a harsh treatment to remove and kill an established biofilm. The reason for this is that bacteria situated in a biofilm structure are protected from established antibacterial treatments. Biofilm formation is believed to involve activation and/or down regulation of a number of genes in response to communication signalling molecules. Gene expression is different in biofilms as compared to free-flowing, planktonic, bacteria.

One approach to the disruption or inhibition of bacterial biofilms is described in U.S. Pat. No. 6,726,898. Compositions and methods of treating periodontal disease are described which employ furanones or furanone derivatives which inhibit or disrupt the glycocalyx matrix of the bacterial biofilm.

Outside the field of antimicrobial compositions Halvorsen et al describe in Synthetic Communications, 2007, 37(7), 1167-1177 oligothiophene compounds for use in materials with non-linear optical properties. Synthesis of various thiophenones is also described in Jakobsen et al in Tetrahedron, 1963, 19, 1867-1882. Hornfeldt and Gronowitz describe in Arkiv foer Kemi, 1963, 21(22), 239-57 the synthesis of further thiophenes without any indication of their utility.

A need exists to find new agents with antimicrobial properties, in particular for use in the prevention or disruption of biofilm formation, which are more effective than those described in the prior art.

### SUMMARY OF THE INVENTION

Accordingly, in a first aspect, the present invention provides an agent comprising the compound according to general formula (I):

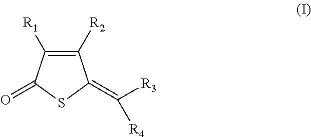

wherein R₁, R₂, R₃ and R₄ are each independently H or a substituent, and wherein at least one of R₁, R₂, R₃ and R₄ is halogen, cyano, cyanate, thiocyanate or C₁-C₆ haloalkyl. In this aspect of the invention, it is preferred that the compound is other than

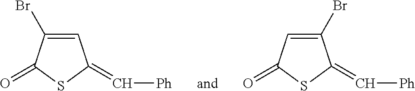

For example, when R₁ or R₂ is Br, it is preferred that one of R₃ and R₄ is not Ph when the other is H.

It has surprisingly been found that compounds according to the present invention act more effectively in inhibition of biofilm formation, as compared with those of the prior art.

In a further aspect, the present invention provides an agent for use in medicine, which agent comprises the compound of general formula (II):

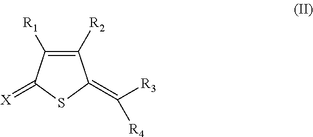

wherein X is O, S, NH or NR′, in which R′ is an optionally substituted C₁-C₆ alkyl group; R₁, R₂, R₃, and R₄ are each independently H or a substituent, and wherein the compound is capable of blocking or interfering with quorum-sensing microbial communication.

In a further aspect the present invention provides an agent for use in medicine, which agent comprises the compound of general formula (II):

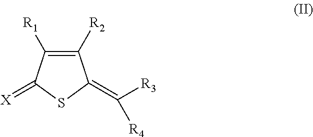

wherein X is O, S, NH or NR′, in which R′ is an optionally substituted C₁-C₆ alkyl group; R₁, R₂, R₃, and R₄ are each independently H or a substituent, and wherein the compound is capable of preventing or inhibiting biofilm formation.

By appropriate selection of substituent groups on the compound of general formula (II), compounds are provided which are capable of blocking or interfering with quorum-sensing microbial communication or preventing or inhibiting biofilm formation. Each of these properties is described in further detail herein, together with tests to demonstrate whether or not the compounds exhibit these properties. Quorum-sensing microbial communication is thought to be mediated by a signalling pathway that is activated as a response to cell density. Such signalling is found in both gram-positive and gram-negative micro-organisms. Perception by bacteria of a quorum-sensing signal occurs at a concentration threshold and it is thought that the bacterial population then responds to the signal. The signal molecules of quorum-sensing systems are thought to be highly specific. It is thought that quorum-sensing systems play a part in biofilm formation. Accordingly, by using the agents of the present invention, quorum-sensing signalling may be blocked or interfered with, thereby interfering with the behaviour of the bacterial population.

A further advantage of interfering with quorum-sensing signalling is that preferred compounds according to the invention which do this do not exert selective pressure on the bacterial population. The bacteria are not killed; instead, their phenotypes are regulated. Accordingly, antimicrobial resistance development is unlikely to result.

In one arrangement, the compound of general formula (II) has a substituent X which is O. Such compounds are relatively straightforward to synthesize and show inhibitory activity towards biofilm formation and towards quorum-sensing.

Each substituent of the compound of general formula (II) may be independently selected from halogen, cyano, cyanate, thiocyanate, alkyl, alkoxy, haloalkyl, alkyl ester, alkylsilyl, alkenyl, alkynyl, aryl, or arylalkyl, which may be substituted or unsubstituted, optionally interrupted by one or more heteroatoms, straight chain or branched chain. Generally, the substituents may have up to six carbon atoms so that the alkyl group is typically a C₁ to C₆ alkyl substituent, the alkoxy substituent is typically a C₁ to C₆ alkoxy substituent and so on. Generally speaking, larger substituent groups may be more likely not to have the activity of blocking or interfering with quorum-sensing microbial communication or preventing or inhibiting biofilm formation. The aryl substituent is preferably a monocyclic aryl, such as phenyl.

It is preferred that each substituent is independently selected from halogen, haloalkyl, alkoxy, alkyl ester, phenyl or

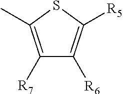

wherein R₅, R₆ and R₇ are each independently H, Br, Cl, OMe, or CHO.

Compounds of high activity have been found where R₄ is a substituent, rather than H. It is further preferred that at least one of R₁, R₃ and R₄ is halogen, cyano, cyanate, thiocyanate or C₁ to C₆ haloalkyl and, more preferably, at least one of R₁ and R₄ is halogen or C₁ to C₆ haloalkyl. R₁ may also be —CH₂—O—CO—(CH₂)₂—COOH or thienyl.

In one arrangement it is preferred that R₁ and R₂ are each independently H or halogen.

In one arrangement it is preferred that R₃ is H, halogen, C₁ to C₆ haloalkyl or phenyl. It is also preferred that R₄ is halogen, C₁ to C₆ haloalkyl or phenyl.

Compounds of high activity have been found where the halogen is Br or I, particularly Br. It is particularly preferred that R₁ is Br. It is also particularly preferred that R₄ is Br. It is particularly preferred that R₂ is H. It is also particularly preferred that R₃ is H. In a further arrangement it is preferred that R₁ and R₄ are each Br and R₂ and R₃ are each H. Alternatively, R₃ and R₄ are each Br and R₁ and R₂ are each H. In a further preferred arrangement, R₁, R₃ and R₄ are each Br and R₂ is H. In a further preferred arrangement R₁ is —CH₂—O—CO—(CH₂)₂—COOH and R₄ is Br. In a further preferred arrangement R₁ is thiophenyl and R₄ is Br. In a further preferred arrangement R₃ is methyl and R₄ is Br. In a further preferred arrangement R₃ is H and R₄ is thiocyanate.

In a further aspect the present invention provides a polymer which comprises a compound as defined herein. An advantage of using a polymeric composition comprising the compound is that surfaces may be treated with a polymer or polymer-forming composition so as to inhibit or prevent biofilm formation thereon. The compound may be incorporated into the polymer as a side chain or in the main chain of the polymer, for example copolymerised with another comonomer to form a copolymer. In one arrangement, the polymer may therefore comprise one or more side chain functional groups comprising the compound wherein the backbone of the polymer is typically a known polymeric backbone such as a polyacrylate, polymethacrylate, polycrotonate, polyvinyl alcohol, polyvinyl acetate, polystyrene, acrylonitrile or siloxane.

In one arrangement, the polymer may be obtainable by polymerising the compound according to general formula (III) with the compound according to general formula (IV).

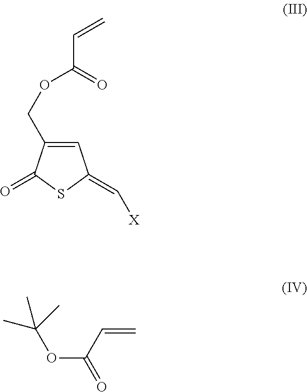

In another arrangement, the compound may be obtainable by polymerising the compound according to general formula (V) with the compound according to general formula (VI):

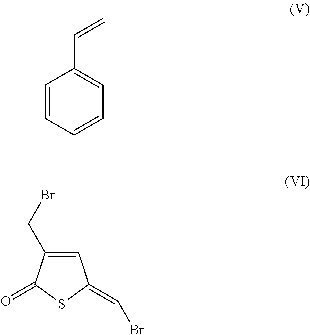

In a further aspect, the present invention provides a process for manufacturing a polymer comprising the compound as defined herein.

Uses of the Agent or Polymer

The agent or polymer of the invention has a wide variety of uses in different fields.

The agent or polymer may be used in medicine in the form described herein or as a pharmaceutically-acceptable salt, ester or prodrug thereof. Pharmaceutically-acceptable salts and esters are well known to those skilled in the pharmaceutical field and they include suitable acid addition salts, base addition salts and esters which are non-toxic to the recipient. A prodrug form may comprise the agent or polymer as a derivative which becomes active only when metabolised by the recipient. Pharmaceutical compositions may be formulated comprising the agent or polymer optionally incorporating a pharmaceutically-acceptable excipient, diluent or carrier, the exact nature of which may be selected according to the intended route of administration. Other ingredients suitable for pharmaceutical use may also be incorporated, as is well known in the pharmaceutical field, such as solvents, buffers, surfactants, preservative agents, and so on.

The agent or polymer according to the invention may be used as an antimicrobial, for example in the prevention or treatment of microbial infection. Microbial infections include bacterial or fungal infections and, in particular, those which involve quorum-sensing microbial communication or biofilm formation. The agent or polymer may interfere with quorum-sensing microbial communication so as to treat or prevent a condition mediated by microbes which are regulated by quorum-sensing communication.

The agent or polymer may be used in conjunction with one or more further antimicrobial agents such as antibiotics or antifungals. In this way, the invention provides a composition comprising an agent or polymer as described herein and one or more further antimicrobial agents as a combined preparation for simultaneous, separate or sequential use in the prevention or treatment of microbial infection. The two components of the combined preparation may be administered separately from one another either at the same time or at separate times. Sequential administration may involve two or more sequential treatments. Where a simultaneous treatment is required, the composition may comprise the components either mixed together or stored separately. The combined preparation may be provided in kit form for convenient use.

The agent, polymer or composition may be used in the treatment of oral conditions, topical infections, respiratory infections, eye infections, ear infections or localised organ infections.

Each of these conditions typically involves biofilm formation and/or microbial quorum-sensing communication. Oral conditions include periodontitis, gingivitis and dental caries. At least some of these conditions need not be addressed using a pharmaceutical composition and may instead be addressed using a personal care product. For example, oral conditions may be treated or prevented using a dentifrice or mouthwash. Topical infections may be treated or prevented using shampoo, soap or deodorant or cosmetic composition. Eye infections may be treated or prevented using a contact lens solution.

The invention further provides a personal care product comprising an agent or polymer as defined herein which is a personal hygiene article, shampoo, soap, deodorant, dentifrice, mouthwash, contact lens solution or cosmetic composition. Such personal care products may be made in a conventional way by incorporating into conventional ingredients the agent or polymer as defined herein.

In a further aspect, the present invention provides an antimicrobial surface cleanser which comprises an agent or polymer as defined herein. The antimicrobial surface cleanser may be formulated for use on an inanimate surface or on the surface of the skin of a human or animal. The antimicrobial surface cleanser may be a disinfectant or a cleaning composition.

In the case of the surface of the skin of an animal or human, it is frequently necessary to ensure that the skin is completely free of microorganisms so that their carriage to other humans or animals is prevented or inhibited.

The antimicrobial surface cleanser may be applied to inanimate surfaces of a very wide variety. Such surfaces include worktops, floors, food preparation tools and equipment surfaces and medical equipment surfaces.

In a further aspect, there is provided a coating composition comprising the agent or polymer as defined herein. In one arrangement, the coating composition is capable of binding covalently to a surface. The coating composition may be in the form of any conventional coating composition such as a paint. In one arrangement, the coating composition comprises a polymer or forms a polymer from suitable reactants on the surface to be treated. In one arrangement, the coating composition comprises the agent covalently linked to the group Si(OR₅)₃, wherein each R₅ is independently substituted or unsubstituted C₁-C₆ hydrocarbyl. The agent may be covalently linked to the group Si(OR₅)₃ by a linker, which linker may comprise a substituted or unsubstituted alkyl, alkenyl, alkynyl, alkylaryl, arylalkyl or aryl linker optionally interrupted by one or more heteroatoms such as O, N and S. In one arrangement the linker comprises —CH₂—O—CO—NH—(CH₂)₃—. The linker preferably attaches to the agent at the R₁ position so that the R₁ substituent is the moiety-linker-Si(OR₅)₃. R₅ is typically ethyl. In one arrangement, the coating composition comprises a compound according to general formula (VII)

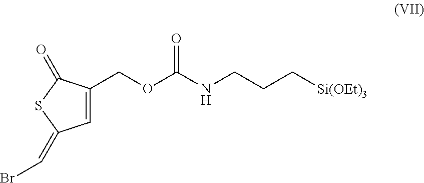

The coating composition may be used as an antibiofouling composition in various applications. Biofouling may occur on marine vessels, submarine installations, pipelines, waterpipes, industrial machines or installations, water systems, cooling towers, heat exchangers, chain lubrication systems, oil or gas platforms, fish farming installations or surfaces, machines, tools or devices used in food production. Biofilm formation is particularly undesirable in these situations. Biocorrosion of the surface may arise over time. By applying a suitable composition to the surface, biofilm formation may be inhibited or prevented. The composition may be painted onto the relevant surface or may be reacted with or polymerised onto the relevant surface. Treatment may be made to the surface in situ or prior to assembly.

In a further aspect, medical devices or implants may be coated with the coating composition of the invention and so coated medical devices or implants are provided. Such devices or implants include catheters, artificial heart valves, surgical pins, pacemaker capsules, prosthetic joints, stents, shunts, endotracheal or gastrointestinal tubes, surgical or dental instruments, surgical suture, dental implants, electrodes, dialysis devices and bandages.

### DETAILED DESCRIPTION OF THE INVENTION

The present invention will now be described in further detail, by way of example only, with reference to the following Examples and accompanying drawings, in which:

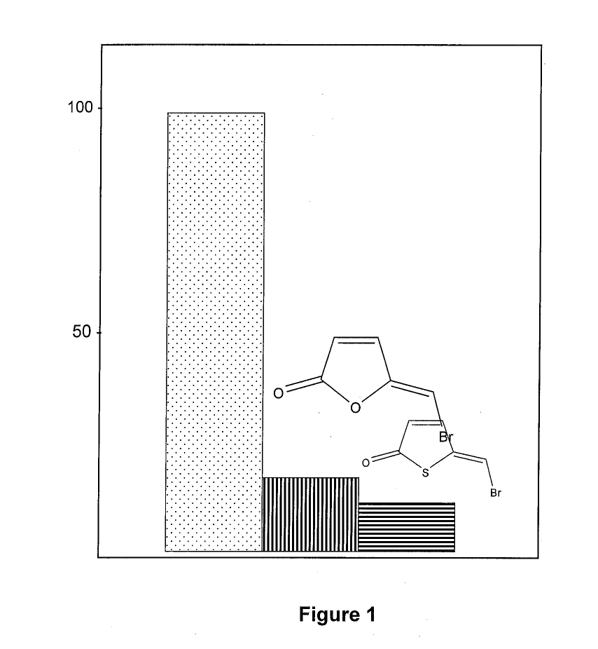

FIG. 1 compares the biofilm inhibitory activity of furanone and thiophenone against Staphylococcus epidermidis

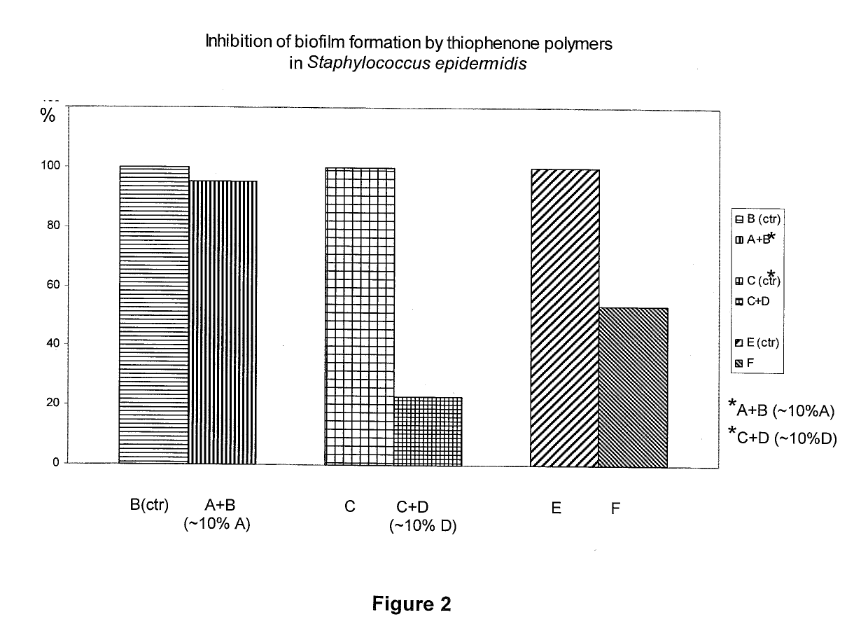

FIG. 2 shows a bar chart comparing biofilm inhibitory activity of polymer coatings according to the invention;

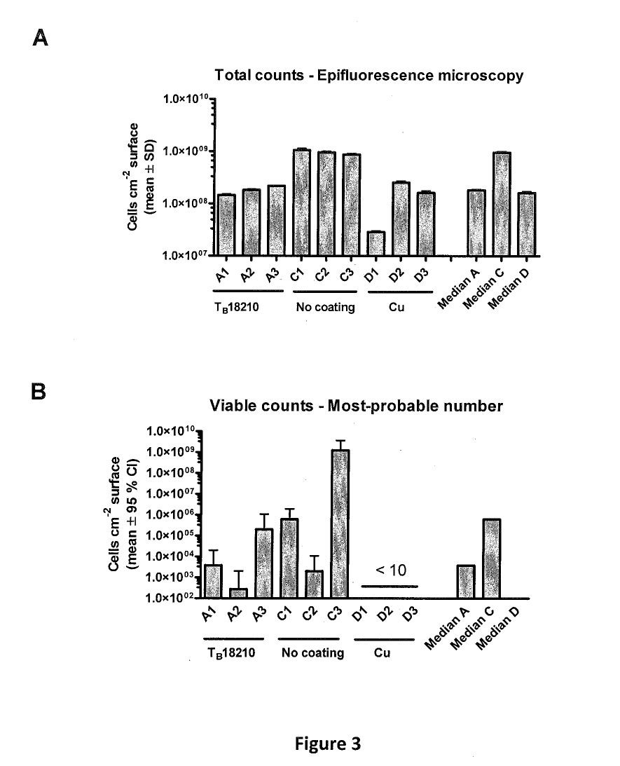

FIG. 3 shows the effect of thiophenone coatings on bacteria desorbed from steel substrates;

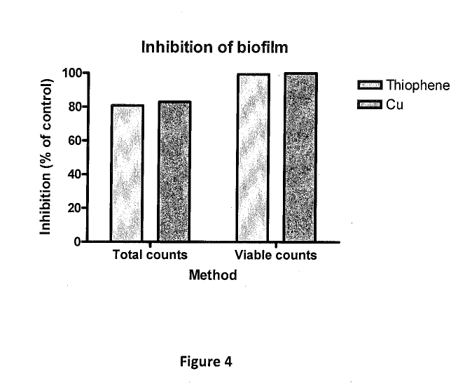

FIG. 4 shows the effect of thiophenone coatings on biofilm growth on steel substrates;

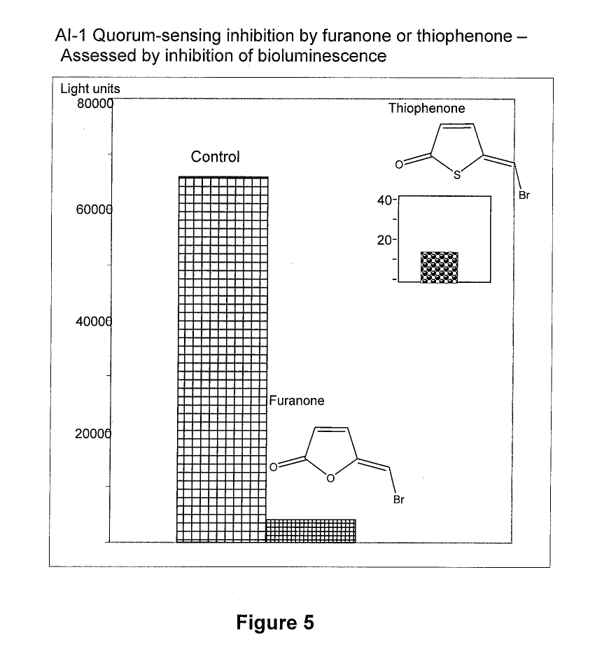

FIG. 5 shows inhibition of AI-1 quorum-sensing by furanone and thiophenone according to the invention; and

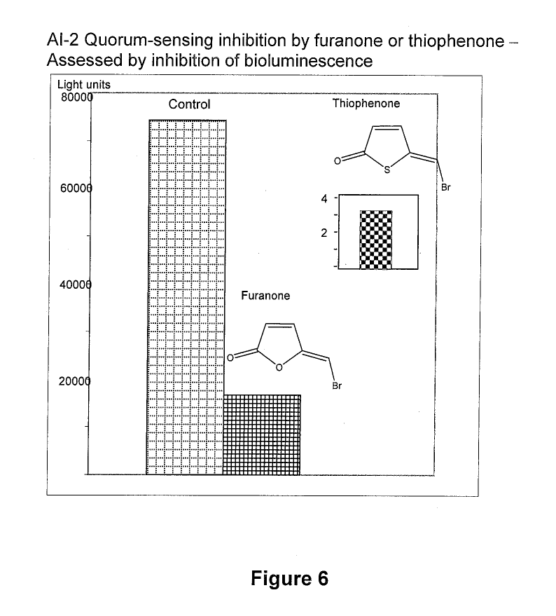

FIG. 6 shows inhibition of AI-2 quorum-sensing by furanone and thiophenone according to the invention.

### EXAMPLES

### Example 1

This example relates to the synthesis of the thiophenones. The compound codes (e.g. Thio101) correspond to those set out in Table 1.

Thio101 and Thio102

(E)- and (Z)-5-Bromomethylenethiophen-2(5H)-one. Acetyl bromide (0.5 mL) was added dropwise at 0° C. to a solution of 5-formyl-2-methoxythiophene¹ (142 mg) in CDCl₃ (1.0 mL) The mixture was stirred at 0° C. for 1.5 h before it was evaporated. The crude product was purified by flash chromatography using hexane/ethyl acetate 5:1 as eluent. Yield (E)-5-bromomethylenethiophen-2(5H)-one: 9 mg. Yield (Z)-5-bromomethylenethiophen-2(5H)-one: 86 mg. The identity of the compounds were confirmed by mass spectrometry and NMR.

Thio103

(Z)-5-Choromethylenethiophen-2(5H)-one. Acetyl chloride (2 mL) was added to 5-formyl-2-methoxythiophene¹ (142 mg). The mixture was stirred at room temperature over night before it was evaporated. The crude product was purified by flash chromatography using hexane/ethyl acetate 5:1 as eluent. Yield: (30 mg. The identity of the compound was confirmed by mass spectrometry and NMR.

Thio104

(Z)-5-Acetyloxymethylenethiophen-2(5H)-one. Acetyl bromide (246 mg) was added dropwise at 0° C. to a solution of 5-formyl-2-methoxythiophene¹ (142 mg) in CDCl₃ (1.0 mL). The mixture was stirred at room temperature over night before it was evaporated. The crude product was purified by flash chromatography using hexane/ethyl acetate 5:1 as eluent. Yield: 40 mg. The identity of the compound was confirmed by mass spectrometry and NMR.

3-Bromo-5-formyl-2-methoxythiophene. 5-Formyl-2-methoxythiophene¹ (2.13 g) was dissolved in dichloromethane (20 mL) at room temperature and N-bromosuccinimide (3.20 g) was added. The mixture was stirred over night. The reaction was diluted with ether (50 mL) and extracted with water. The organic phase was dried (MgSO₄), filtered and evaporated. The crude product was purified by flash chromatography (gradient elution: 0-25% EtOAc in hexanes). Yield: 2.53 g. The identity of the compound was confirmed by mass spectrometry and NMR.

Thio105

(Z)-3-Bromo-5-bromomethylenethiophen-2(5H)-one. Acetyl bromide (3.0 mL) was added. to a solution of 3-bromo-5-formyl-2-methoxythiophene (882 mg) in dichloromethane (5 mL) The reaction was stirred for 48 h before it was diluted with ether (40 mL) and extracted with NaOH (1.0M, aq) and water. The organic phase was dried (MgSO₄), filtered and evaporated. The crude product was purified by flash chromatography (gradient elution: 0-20% EtOAc in hexanes). Yield: 301 mg. The identity of the compound was confirmed by mass spectrometry and NMR.

Thio106 and Thio108

5-Dibromomethylenethiophen-2(5H)-one and 3-bromo-5-dibromomethylenethiophen-2(5H)-one. Bromine (0.5 mL, 2M in CCl₄) was added dropwise at 0° C. to a solution of (Z)-5-bromomethylenethiophen-2(5H)-one (40 mg) in CDCl₃ (1 mL). The mixture was stirred at room temperature for 4 h before another portion of bromine (0.2 mL) was added. Stirring was continued for 2 h before the mixture was evaporated and diisopropylethylamine (44 mg) added. The mixture was stirred for 2 h before diethyl ether was added and the organic phase washed with aqueous HCl (1M). The solution was dried (MgSO₄) and evaporated. The crude product was purified by flash chromatography using hexane/ethyl acetate 8:1 as eluent. Yield 3-bromo-5-dibromomethylenethiophen-2(5H)-one: 16 mg. Yield 5-dibromomethylenethiophen-2(5H)-one: 2 mg. The identity of the compounds were confirmed by mass spectrometry and NMR.

Thio 107

(Z)-5-(2,2-Dibromoethylidene)thiophen-2(5H)-one. Acetyl bromide (1 mL) was added dropwise at 0° C. to a solution of 2-(2,2-dibromovinyl)-5-methoxythiophene (230 mg) in CDCl₃ (2.0 mL) The mixture was stirred at room temperature for 5 h before it was evaporated. Ether was added and the solution washed with aqueous NaHCO₃ and brine. The dried solution (MgSO₄) was evaporated and the crude product was purified by flash chromatography using hexane/ethyl acetate 5:1 as eluent. Yield: 105 mg. The identity of the compound was confirmed by mass spectrometry and NMR.

Thio109

(Z)-5-Benzylidenethiophen-2(5H)-one. Acetyl bromide (0.035 mL) was added at 0° C. to a solution of (5-methoxythiophen-2-yl)(phenyl)methanol³ (103 mg) in CDCl₃ (1.0 mL) The mixture was stirred at 0° C. for 30 min before diethyl ether was added and the solution washed with aqueous NaHCO₃. The crude product was purified by flash chromatography using hexane/ethyl acetate 5:1 as eluent. Yield: 44 mg. The identity of the compound was confirmed by mass spectrometry and NMR.

Thio110

(Z)-3-Bromo-5-benzylidenethiophen-2(5H)-one. Bromine (0.05 mL, 2M in CCl₄) was added at 0° C. to a solution of (Z)-5-benzylidenethiophen-2(5H)-one (16 mg) in CDCl₃ (1 mL). The mixture was stirred for 24 h at room temperature before it was evaporated and dissolved in CH₂Cl₂ (1 mL). Diisopropylethylamine (39 mg) was added and the mixture stirred for 2 h before washing with HCl (1 M). The crude product was purified by flash chromatography using hexane/ethyl acetate 5:1 as eluent. Yield: 16 mg. The identity of the compound was confirmed by mass spectrometry and NMR.

Thio111 and Thio112

(E)- and (Z)-5-Bromo(phenyl)methylenethiophen-2(5H)-one. Acetyl bromide (1.5 mL) was added dropwise at room temperature to a solution of 5-benzoyl-2-methoxythiophene² (100 mg) in CH₂Cl₂ (1.0 mL). The mixture was stirred at room temperature for 8 d and under reflux for 4 h before it was evaporated. The crude product was purified by flash chromatography using hexane/ethyl acetate 8:1 as eluent. Yield bromo(phenyl)methylenethiophen-2(5H)-one: 38 mg. Yield (Z)-5-bromo(phenyl)methylenethiophen-2(5H)-one: 17 mg The identity of the compounds were confirmed by mass spectrometry and NMR.

Thio113

(Z)-3-Bromomethyl-5-bromomethylenethiophen-2(5H)-one. 3 -Chloromethyl-5-formyl-2-methoxythiophene (400 mg) was dissolved in dichloromethane (4 mL) at room temperature and acetyl bromide (1.6 mL) was added. The mixture was stirred for 96 h at room temperature before it was diluted with ether (20 mL) and extracted with NaOH (1.0M, aq) and water. The organic phase was dried (MgSO₄), filtered and evaporated. The crude product was purified by flash chromatography (gradient elution: 0-20% EtOAc in hexanes). Yield: 264 mg. The identity of the compound was confirmed by mass spectrometry and NMR.

Thio115

(Z)-3-Hydroxymethyl-5-bromomethylenethiophen-2(5H)-one. (Z)-3-Bromomethyl-5-bromomethylenethiophen-2(5H)-one (970 mg) was dissolved in 20 mL acetone/water (9:1) at room temperature in a round bottom flask covered with aluminium foil before silver triflate (2.62 g) was added. The mixture was stirred for 24 h at room temperature before it was diluted with ether (50 mL) and extracted with brine. The organic phase was dried (MgSO₄), filtered and evaporated. The crude product was purified by flash chromatography (gradient elution: 0-40% EtOAc in hexanes). Yield: 550 mg. The identity of the compound was confirmed by mass spectrometry and NMR.

Thio202

2-(2,2-Dibromovinyl)-5-methoxythiophene: Tetrabromomethane (0.70 g) and triphenylphosphine (1.0 g) were successively added to a solution of 5-formyl-2-methoxythiophene¹ (282 mg) in dichloromethane (10 mL). After stirring for 5 min was another portion of triphenylphosphine (0.2 g) was added and the mixture stirred at 0° C. for 30 min. The mixture was then filtered through a short pad of silica gel and purified by flash chromatography using hexane/ethyl acetate 10:1 as eluent. Yield: 230 mg. The identity of the compound was confirmed by mass spectrometry and NMR.

Thio301

### (Z)-5-(Thiophen-2-ylmethylene)thiophen-2(5H)-one⁴

Thio302

### (Z)-5-((5-Bromothiophen-2-yl)methylene)thiophen-2(5H)-one⁴

Thio304

### (Z)-5-((5-Methoxythiophen-2-yl)methylene)thiophen-2(5H)-one⁴

Thio305

(Z)-5-((3,4-Dibromo-5-methoxythiophen-2-yl)methylene)thiophen-2(5H)-one. Bromine (0.035 mL, 2M in CCl₄) was added at 0° C. to a solution of (Z)-5-((5-methoxythiophen-2-yl)methylene)thiophen-2(5H)-one⁴ (12 mg) in CDCl₃ (1 mL). The mixture was stirred for 1 h at 0° C. before ether was added. The organic solution was washed with aqueous thiosulfate, dried (MgSO₄) and evaporated. Yield : 15 mg. The identity of the compound was confirmed by NMR.

3-Chloromethyl-5-formyl-2-methoxythiophene. 5-Formyl-2-methoxythiophene (141 mg) was dissolved in dichloromethane (1 mL) at 0° C. before chloromethyl ethyl ether (0.48 mL) was added followed by TiCl₄ (0.17 mL) The mixture was stirred for 2 hours at room temperature before was diluted with dichloromethane (20 mL) and extracted with water and brine. The organic phase was dried (MgSO₄), filtered and evaporated. The crude product was purified by flash chromatography (gradient elution: 0-25% EtOAc in hexanes). Yield: 83 mg. The identity of the compound was confirmed by mass spectrometry and NMR.

(5-Bromomethylene-2-oxo-2,5-dihydrothiophen-3-yl)methyl acrylate and (5-chloromethylene-2-oxo-2,5-dihydrothiophen-3-yl)methyl acrylate. (Z)-3-Hydroxymethyl-5-bromomethylenethiophen-2(5H)-one (220 mg) was dissolved in dichloromethane (2 mL) before acryloyl chloride (180 mg) and triethylamine (0.15 g) were added. The mixture was stirred for 1 h at room temperature before it was diluted with ether (25 mL) and extracted with water. The organic phase was dried (MgSO₄), filtered and evaporated. The crude product was purified by flash chromatography (gradient elution: 0-20% EtOAc in hexanes) to give a 1:1 mixture of the title compounds. Yield: 213 mg. The identity of the compounds were confirmed by mass spectrometry and NMR.

Benzyl 3-(triethoxysilyl)propylcarbamate To benzyl alcohol (2.16 g) was (3-isocyanatopropyl)triethoxysilane (5.2 mL) added and the mixture stirred at 85° C. for 3 h. The reaction was dried in vacuo. The crude product was purified by flash chromatography (gradient elution: 0-10% EtOAc in hexanes). Yield: 6.53 g. The identity of the compound was confirmed by NMR.

(Z)-(5-bromomethylene-2-oxo-2,5-dihydrothiopen-3-yl)methyl 3-(triethoxysilyl)propylcarbamate. A mixture of (Z)-3-Hydroxymethyl-5-bromomethylenethiophen-2(5H)-one (44 mg) and triethoxy(3-isocyanatopropyl)silane (248 mg) in dry toluene (1 mL) was heated at 60° C. over night. The solvent was evaporated off and the crude product was purified by flash chromatography using hexane/ethyl acetate 2:1 as eluent. Yield 40 mg. The identity of the compound was confirmed by NMR.

### REFERENCES

1) Profft, Elmar, Justus Liebigs Annalen der Chemie (1959), 622 196-200.
2) Pearson, D. E.; Buehler, Calvin A. Synthesis (1972), (10), 533-42.
3) Lavrushin, V. F.; Nikitchenko, V. M.; Trusevich, N. D.; Pedchenko, N. F.; Kanate, B.; Pivnenko, N. S.; Pogonina, R. I. Khar'k. Gos. Univ. im. Gor'kogo, Kharkov, USSR. Editor(s): Gal'pern, G. D. Tezisy Dokl. Nauchn. Sess. Khim. Tekhnol. Org. Soedin. Sery Sernistykh Neftei, 13th (1974), 182-3.
4) H. Halvorsen, H. Hope and J. Skramstad, Synth. Commun. 37 (2007) 1167-1177

### Example 2a

This example describes the effect of thiophenones on biofilm formation by various bacteria. Biofilm formation was measured according to a static biofilm model and according to a shaking biofilm model and it was shown that the various thiophenones tested were found to inhibit or prevent biofilm formation.

According to the static biofilm model, a given thiophenone 200 μmol/L was dissolved in 500 μl absolute ethanol and applied to wells of a standard 24 well microtiter plate. The ethanol was evaporated from the wells in a laminar air sterile work bench at room temperature so as to leave a coating of the thiophenone in the well. A sample of bacteria was then added to the well and incubated for a given period of time. After incubation, the percentage of bacteria remaining was assessed by safranine staining of the biofilm. Bound safranine was released by acetic acid and optical density was measured in Synergy HT Multi-Detection Microplate Reader and compared to a control. The results are set out in Table 1 below.

According to the shaking biofilm model, the microtiter plates were shaken (200 rpm) in a Minitron Incubator Shaker during biofilm formation.

Table 1 shows the results of tests on various thiophene structures in this example. The structure and name of each thiophene is given, together with the bacteria tested. The percentage of bacteria remaining in the biofilm is shown, together with the time of incubation.

It may be concluded from these results that Thiophenone inhibits biofilm formation by various bacteria. The thiophenone effect is mediated through interference with quorum-sensing communication via AI-1 and AI-2. The thiophenone is more effective than furanone (FIGS. 1-4).

TABLE 1
Effect of thiophenones on biofilm formation by various bacteria

| | | | | Static biofilm model | | Shaking biofilm model | |
|---|---|---|---|---|---|---|---|
| | | | | % | Time | % | Time |
| Structure | Name | | | Remaining | incubation | Remaining | incubation |
| 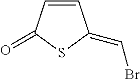 | Thio101 (Z)-5-(bromomethylene) thiophen-2(5H)-one Chemical Formula: C₅H₃BrOS Molecular Weight: 191.05 | S. epidermidis S. epidermidis E. faecalis E. faecalis V. harveyi V. harveyi V. harveyi V. harveyi Pseudo- alteromonas Pseudo- alteromonas | coating in medium coating in medium coating in medium | 28 66 32 20 9 52 36 | 6 h 4 h 4 h 4 h 4 h 4 h 4 h | 13 15 54 56 | 6 h 20 h 8 h 18 h |
| 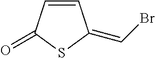 | Thio102 (E)-5-(bromomethylene) thiophen-2(5H)-one Chemical Formula: C₅H₃BrOS Molecular Weight: 191.05 | S. epidermidis S. epidermidis | | 95 | 6 h | 91 103 | 6 h 20 h |
| 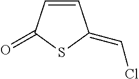 | Thio103 (Z)-5-(chloromethylene) thiophen-2(5H)-one Chemical Formula: C₅H₃ClOS Molecular Weight: 146.59 | S. epidermidis S. epidermidis | | 32 | 6 h | 92 96 | 6 h 20 h |
| 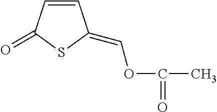 | Thio104 (Z)-(5-oxothiophen-2(5H)- ylidene)methyl acetate Chemical Formula: C₇H₆O₃S Molecular Weight: 170.19 | S. epidermidis S. epidermidis | | 34 | 6 h | 106 103 | 6 h 20 h |
| 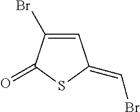 | Thio105 (Z)-3-bromo-5- (bromomethylene)thiophen- 2(5H)-one Chemical Formula: C₅H₂Br₂OS Molecular Weight: 269.94 | S. epidermidis S. epidermidis | | | | 20 18 | 6 h 20 h |
| 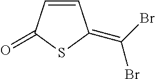 | Thio106 5-(dibromomethylene)thiophen- 2(5H)-one Chemical Formula: C₅H₂Br₂OS Molecular Weight: 269.94 | S. epidermidis S. epidermidis | | | | 11 27 | 6 h 20 h |
| 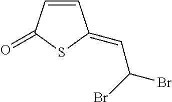 | Thio107 (Z)-5-(2,2-dibromoethylidene) thiophen-2(5H)-one Chemical Formula: C₆H₄Br₂OS Molecular Weight: 283.97 | S. epidermidis S. epidermidis | | | | 77 93 | 6 h 20 h |
| 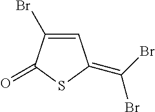 | Thio108 3-bromo-5- (dibromomethylene)thiophen- 2(5H)-one Chemical Formula: C₅HBr₃OS Molecular Weight: 348.84 | S. epidermidis S. epidermidis | | | | 1 9 | 6 h 20 h |
| 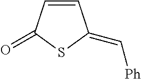 | Thio109 (Z)-5-benzylidenethiophen- 2(5H)-one Chemical Formula: C₁₁H₈OS Molecular Weight: 188.25 | S. epidermidis S. epidermidis | | | | 98 107 | 6 h 20 h |
| 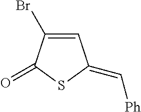 | Thio110 (Z)-5-benzylidene-3- bromothiophen-2(5H)-one Chemical Formula: C₁₁H₇BrOS Molecular Weight: 267.14 | S. epidermidis S. epidermidis | | | | 30 27 | 6 h 20 h |
| 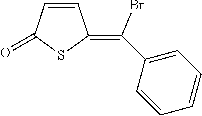 | Thio111 (E)-5-(bromo(phenyl)methylene) thiophen-2-(5H)-one Chemical Formula: C₁₁H₇BrOS Molecular Weight: 267.14 | S. epidermidis S. epidermidis | | | | 63 50 | 6 h 20 h |
| 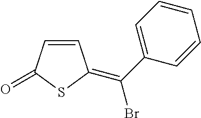 | Thio112 (Z)-5-(bromo(phenyl)methylene) thiophen-2(5H)-one Chemical Formula: C₁₁H₇BrOS Molecular Weight: 267.14 | S. epidermidis S. epidermidis | | | | 63 59 | 6 h 20 h |
| 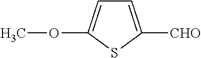 | Thio201 5-methoxythiophene-2- carbaldehyde Chemical Formula: C₆H₆O₂S Molecular Weight: 142.18 | S. epidermidis S. epidermidis | | 56 | 6 h | 81 101 | 6 h 20 h |
| 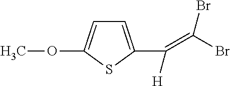 | Thio202 2-(2,2-dibromovinyl)- 5-methoxythiophene Chemical Formula: C₇H₆Br₂OS Molecular Weight: 297.99 | S. epidermidis S. epidermidis | | | | 94 103 | 6 h 20 h |
| 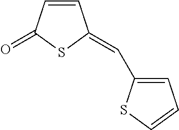 | Thio301 (Z)-5-(thiophen-2-ylmethylene) thiophen-2(5H)-one Chemical Formula: C₉H₆OS₂ Molecular Weight: 194.27 | S. epidermidis S. epidermidis | | | | 80 98 | 6 h 20 h |
| 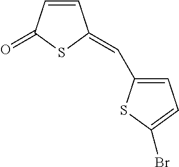 | Thio302 (Z)-5-((5-bromothiophen-2-yl) methylene)thiophen-2(5H)-one Chemical Formula: C₉H₅BrOS₂ Molecular Weight: 273.17 | S. epidermidis S. epidermidis | | | | 98 101 | 6 h 20 h |
| 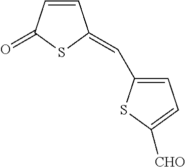 | Thio303 (Z)-5-((5-oxothiophen-2(5H)- ylidene)methyl)thiophene- 2-carbaldehyde Chemical Formula: C₁₀H₆O₂S₂ Molecular Weight: 222.28 | S. epidermidis S. epidermidis | | | | 95 91 | 6 h 20 h |
| 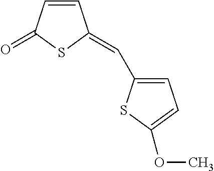 | Thio304 (Z)-5-((5-methoxythiophen-2- yl)methylene)thiophen- 2(5H)-one Chemical Formula: C₁₀H₈O₂S₂ Molecular Weight: 224.30 | S. epidermidis S. epidermidis | | | | 72 94 | 6 h 20 h |
| 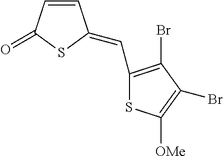 | Thio305 (Z)-5-((3,4-dibromo-5- methoxythiophen-2- yl)methylene)thiophen- 2(5H)-one Chemical Formula: C₁₀H₆Br₂O₂S₂ Molecular Weight: 382.09 | S. epidermidis S. epidermidis | | | | 66 101 | 6 h 20 h |
| 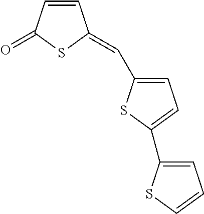 | Thio306 (Z)-5-(2,2′-bithiophen-5- ylmethylene) thiophen-2(5H)-one Chemical Formula: C₁₃H₈OS₃ Molecular Weight: 276.40 | S. epidermidis | | 67 | 6 h | | |
| 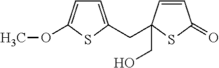 | Thi401 5-(hydroxymethyl)-5-((5- methoxythiophen-2- yl)methyl)thiophen- 2(5H)-one Chemical Formula: C₁₁H₁₂O₃S₂ Molecular Weight: 256.34 | S. epidermidis S. epidermidis | | 51 | 6 h | 80 94 | 6 h 20 h |

### Example 2b

This example describes the effect of further thiophenes on biofilm formation and planktonic growth by various bacteria.

Planktonic growth was determined in “Low Binding Plates” in which the bacteria form minimal amounts of biofilm The quantity is determined by optical density measurements at 600 nm.

Biofilm formation was measured in static cultures in wells of microtiter plates for S. epidermidis, or on “peggs” according to the Calgary method (The Calgary biofilm devices: New technology for rapid determination of antibiotic susceptibilities of bacterial biofilms. Ceri et al. J Clin Microbiol 1999:37:1771-1776) for V. harveyi. In both cases the safranin staining method was applied and optical density was measured at 530 nm for quantification of biofilm mass.

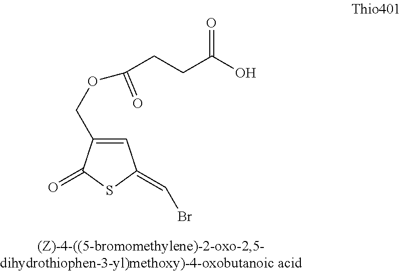

This compound was synthesized as follows:

Hünig's base (155 mg, 1.2 mmol) and DMAP (catalytic amount, ˜10 mg) was dissolved in 2 mL DCM and added to a solution of succinic anhydride (120 mg, 1.2 mmol) and (Z)-5-(bromomethylene)-3-(hydroxymethyl)thiophen-2(5H)-one (0.22 g, 1.0 mmol) in 4 mL DCM at room temperature. The reaction mixture was stirred for 30 minutes, diluted with 25 mL DCM and washed with 3*5 mL water. The combined aqueous phases were extracted with 2*10 mL ether. The combined organic phases were dried over MgSO₄, filtrated and the solvents were removed in vacuo. The residue was dissolved in a small amount of THF and ether(1:2), and the product was precipitated by addition of pentane. The solution was filtered, leaving a yellow solid. Yield: 220 mg. The identity of the compound was confirmed by mass spectrometry and NMR.

Biofilm formation by V. harveyi was measured using the Calgary method. There was a 17% reduction at a thiophenone concentration of 5 μM, 70% reduction at 50 μM and 85% reduction at 100 μM. This represents a strong effect on V. harveyi biofilm reduction. However a weak effect on planktonic bacteria of 18% reduction at 100 μM was observed.

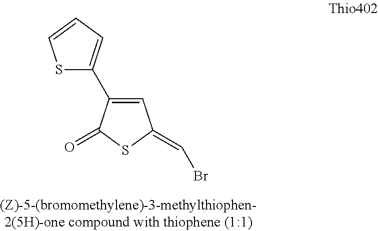

### Synthesis of 2′-methoxy-2,3′-bithiophene-5′-carbaldehyde

4-bromo-5-methoxythiophene-2-carbaldehyde (0.22 g, 1 mmol), tributyl(thiophen-2-yl)stannane (0.75 g, 2 mmol), PdCl₂(PhCN)₂(38 mg, 0.1 mmol) and PPh₃ (79 mg, 0.3 mmol) were dissolved in N,N-dimethylformamide(3 mL) and stirred at 50° C. for 24 h. The reaction mixture was cooled to room temperature, diluted with 25 mL ether, and washed with 3*10 mL water. The combined aqueous phases was extracted with 10 mL ether, and the combined organic phases were dried over MgSO₄, filtrated and the solvents removed in vacuo. The product was purified by flash column chromatography on silica (0-20% EtOAc in hexanes). Yield: 187 mg. The identity of the compound was confirmed by mass spectrometry and NMR.

### Synthesis of (Z)-5-(bromomethylene)-3-(thiophen-2-yl)thiophen-2(5H)-one

Acetylbromide (0.56 mL, 7.5 mmol) was added dropwise to a solution of 2′-methoxy-2,3′-bithiophene-5′-carbaldehyde (0.11 g, 0.5 mmol) in 6 mL DCM at 0° C. The mixture was heated to room temperature and stirred for 3 days. The mixture was diluted with 10 mL ether, washed with 10 mL NaOH (aq, 1M) and 2*5 mL water. The combined aqueous phases were extracted with 25 mL DCM, and the combined organic phases were dried over MgSO₄, filtrated and the solvents were removed in vacuo. The product mixture was purified by flash column chromatography on silica (0-15% EtOAc in hexanes). Yield: 44 mg. The identity of the compound was confirmed by mass spectrometry and NMR.

Using the Calgary method, a V. harveyi biofilm reduction of 40 to 50% was observed in the presence of the thiophenone at a concentration of from 40 to 100 μM. This compound is therefore less effective than Thio401. A 30% reduction in planktonic bacteria was observed at 100 μM.

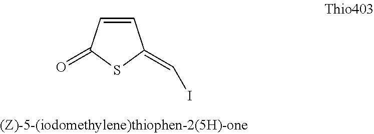

This compound was synthesized as follows:

(Z)-5-(Bromomethylene)thiophen-2(5H)-one (103 mg) in acetone (2 mL) was added to a solution of sodium iodide (530 mg) in acetone (3 mL). The mixture was stirred for 3 days at room temperature before ether and water was added. The aqueous phase was extracted with ether and the combined organic phase was dried (MgSO₄) and evaporated. The crude product was purified by flash chromatography (hexane/EtOAc 5:1). Yield 97 mg. The identity of the compound was confirmed by mass spectrometry and NMR.

This compound showed a biofilm reduction of 46% at 5 μM, 80% at 10 μM and 95% at 15 μM when tested against V. harveyi bacteria. This compound is therefore strongly effective against biofilm formation. It is also strongly effective on planktonic bacterial growth showing a 70 to 80% reduction at 15 μM to 100 μM for V. harveyi.

The same compound was tested against S. epidermis and found to be less effective. Biofilm reduction was 50% at 50 μM and 64% at 100 μM. Planktonic bacterial growth reduction was 50% at 50 μM and 63% at 100 μM.

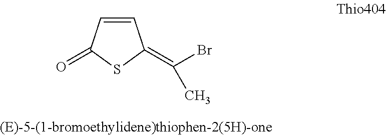

This compound was synthesized as follows:

Acetyl bromide (0.017 mL) was added at room temperature to a solution of 5-acetyl -2-methoxythiophene^{REF} (21 mg) in CDCl₃ (1.0 mL) The mixture was stirred at room temperature for 24 h befor another portion of acetyl bromide (0.15 mL) was added. The mixture was stirred for 3 d before it was evaporated and purified by flash chromatography (hexane/EtoAc 5:1). Yield E-isomer: 11 mg. Yield Z-isomer: 5 mg. The identity of the compounds were confirmed by mass spectrometry and NMR.

REF

Sice, Jean, Journal of the American Chemical Society (1953), 75 3697-700

This compound was tested on V. harveyi for biofilm reduction and showed 70 to 80% reduction at 50 to 100 μM, which may be considered a moderate effect. A relatively minor effect on planktonic growth was observed with 40 to 55% reduction at 50 to 100 μM.

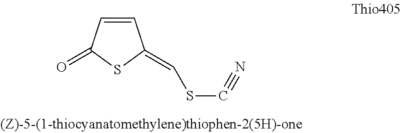

This compound was synthesized as follows:

Ammonium isothiocyanate (34 mg) was added to a solution of (Z)-5-(1-bromomethylidene)thiophen-2(5H)-one (22 mg) in acetone. The mixture was stirred at room temperature for 1 h before before ether and water was added. The aqueous phase was extracted with ether and the combined organic phase was dried (MgSO₄) and evaporated. The identity of the compound was confirmed by mass spectrometry and NMR.

This compound had a strong effect on biofilm reduction and planktonic growth in S. epidermis. A biofilm reduction of 90 to 100% was observed at concentrations from 25 μM. Planktonic growth was reduced by 70% at thiophenone concentrations from 25 μM.

The compound was also found to have a strong effect on V. harveyi biofilm reduction showing a 90% reduction at 25 μM. Planktonic reduction for V. harveyi was 50% at 25 μM.

### Example 2c (Comparative Example)

This example follows the same methodology as Example 2b. However, the substituent groups on the thiophenones are unsuitable to render the compound capable of blocking or interfering with quorum-sensing microbial communication or preventing or inhibiting biofilm formation.

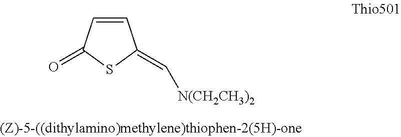

This compound was synthesized as follows:

A mixture of diethyl amine (47 mg) in CDCl₃ (1.5 mL) was slowly added to a solution of (Z)-5-(1-bromomethylidene)thiophen-2(5H)-one (46 mg) in CDCl₃ (1.5 mL) at 0° C. The mixture was stirred for 2.5 h before it was evaporated. The crude product was purified by flash chromatography (CHCl₃/MeOH 20:1). Yield: 58 mg. The identity of the compound was confirmed by mass spectrometry and NMR.

No effect on V. harveyi biofilm or planktonic growth.

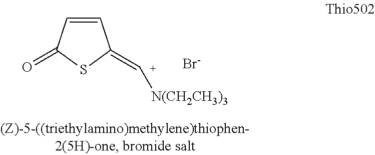

This compound was synthesized as follows:

A mixture of triethyl amine (10 mg) in CDCl₃ (1 mL) was slowly added to a solution of (Z)-5-(1-bromomethylidene)thiophen-2(5H)-one (14 mg) in CDCl₃ (1 mL) at 0° C. The mixture was stirred at room temperature for 24 h before it was evaporated. The identity of the compound was confirmed by NMR.

No effect on S. epidermis biofilm or planktonic growth.

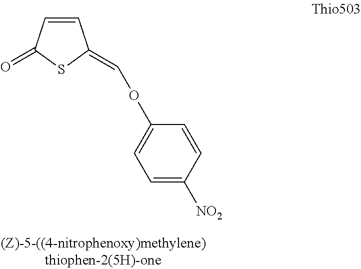

This compound was synthesized as follows:

A mixture of (Z)-5-(1-bromomethylidene)thiophen-2(5H)-one (20 mg), p-nitrophenol (58 mg) and triethyl amine (42 mg) in CDCl₃ (1.5 mL) was stirred at room temperature for 18 h before it was evaporated. The crude product was purified by flash chromatography (CH₂Cl₂). Yield: 21 mg. The identity of the compound was confirmed by mass spectrometry and NMR.

No effect on S. epidermis biofilm or planktonic growth.

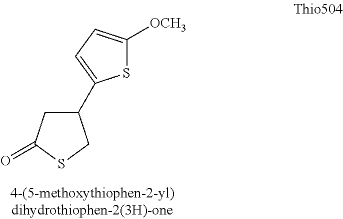

This compound was synthesized as follows:

Dry HCl was bubbled through a solution of 2-methoxythiophene (224 mg) in CDCl₃ (2 Ml) at 0° C. for ca 2 min. The mixture was left at room temperature over night, evaporated and purified by Flash chromatography. hexane/EtOAc 5:1. Yield 123 mg. The identity of the compound was confirmed by mass spectrometry and NMR

No effect on V. harveyi biofilm or planktonic growth.

### Example 3

This example relates to the synthesis of polymeric thiophenones and thiophenones containing functional groups for adhesion to surfaces.

Formation of a Co-Polymer from Compound 1 and Styrene:

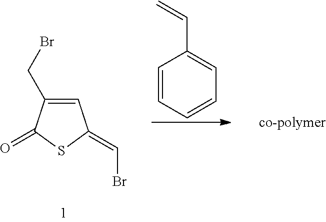

320 mg of compound 1, 2.88 g of styrene and 160 mg of AIBN (Azo Bis Isobutyronitrile) were added to 6 mL of toluene and degassed with argon for 30 minutes at rt. The solution was then stirred at 70° C. for 24 h., cooled to room temperature, and added to 25 mL pentane. The precipitate was washed two times with pentane and dried under high vacuum at room temperature overnight.

The copolymer formed in this way has the predicted general formula

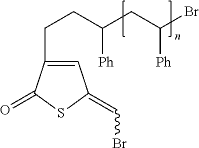

in which n is an integer. The thiophene agent forms an end group or side chain.

### Formation of a Styrene Polymer (Comparative Example)

10 g styrene was added to 10 mL of toluene and 100 mg of AIBN. The solution was degassed with argon for 30 min at room temperature. The solution was stirred for 3 h at 70° C., cooled to room temperature, and added to 40 mL of pentane. The precipitate was washed two times with pentane and dried under high vacuum at room temperature overnight.

Formation of a Co-Polymer from Compound 2) and Tert-Butyl Acrylate:

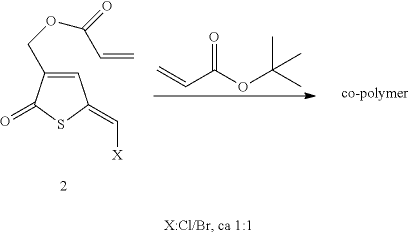

240 mg of compound 2, 2.16 g of tert-butyl acrylate and 120 mg of AIBN were added to 5 mL of toluene and degassed with argon for 30 minutes at room temperature. The solution was stirred at 70° C. for 24 h., cooled to room temperature, and added to 25 mL pentane. The precipitate was washed two times with pentane and dried under high vacuum at room temperature overnight.

The monomer units of the copolymer formed in this way may be linked together as shown below:

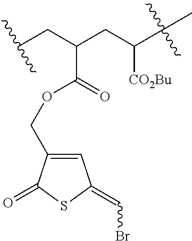

### Formation of a Tert-Butyl Acrylate Polymer (Comparative Example)

10 g tert-butyl acrylate was added to 10 mL of toluene and 100 mg of AIBN. The solution was degassed with argon for 30 min at room temperature. The solution was stirred for 1 h at 70° C., cooled to room temperature, and added to 40 mL of pentane. The residue was washed two times with pentane and dried under high vacuum at room temperature overnight.

### Formation of Benzyl 3-(triethoxysilyl)propylcarbamate (Compound E)

To benzyl alcohol (2.16 g) was (3-isocyanatopropyl)triethoxysilane (5.2 mL) added and the mixture stirred at 85° C. for 3 h. The reaction was dried in vacuo. The crude product was purified by flash chromatography (gradient elution: 0-10% EtOAc in hexanes). Yield: 6.53 g. The identity of the compound was confirmed by NMR.

### Formation of (Z)-(5-bromomethylene-2-oxo-2,5-dihydrothiopen-3-yl)methyl 3-(triethoxysilyl)propylcarbamate (Compound F):

A mixture of (Z)-3-Hydroxymethyl-5-bromomethylenethiophen-2(5H)-one (44 mg) and triethoxy(3-isocyanatopropyl)silane (248 mg) in dry toluene (1 mL) was heated at 60° C. over night. The solvent was evaporated off and the crude product was purified by flash chromatography using hexane/ethyl acetate 2:1 as eluent. Yield: 40 mg. The identity of the compound was confirmed by NMR

### Example 4

This example relates to the effects of surface coatings on biofilm formation by bacteria.

The materials of Example 3 were tested in a static biofilm model as follows.

Triplicate samples of a coating composition were applied to a glass vial in accordance with Table 2 set out below where each vial is given a number. Vials 1 to 12 are each loaded with 13 mg of the indicated material, followed by the addition of 1.0 mL dichloromethane. The dichloromethane was slowly evaporated under atmospheric pressure to leave a coating on the internal walls of the receptacles. Vials 13 to 18 were each loaded by adding 10 mg of the indicated material pre-dissolved in 10 mL dichloromethane. These vials were heated in an oven at 125° C. for 24 hours. To remove any residues of the dichloromethane solvent, all vials were dried at high vacuum (0.5 mmHg) for 2 hours at room temperature.

TABLE 2

| Glass Vial | Material |
|---|---|
| 1, 2 and 3 | Copolymer of A and B (~10% by weight of A and ~90% |
| | by weight of B) |
| 4, 5 and 6 | Polymer of B |
| 7, 8 and 9 | Polymer of C |
| 10, 11 and 12 | Copolymer of C and D (~10% by weight of D and ~90% |
| | by weight of C) |
| 13, 14 and 15 | E |
| 16, 17 and 18 | F |

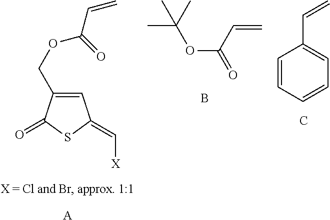

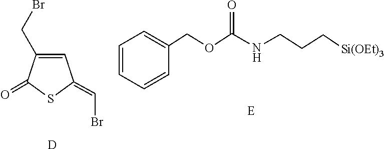

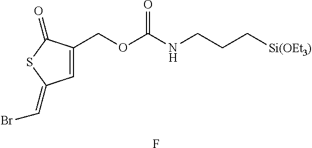

FIG. 2 shows the results of incubating bacteria S. epidermidis over a time period of 4 hours. The biofilm was assessed as described in Example 2.

These results show that thiophene polymers have inhibitory activity in relation to biofilm formation.

The results also show that thiophenes according to the invention may be covalently bound to a surface so as to inhibit biofilm formation. It is possible in the example of the copolymer of C and D that bacteria are killed in addition to the inhibition of biofilm formation. Similar results were obtained in a static biofilm model where S. epidermis bacteria were incubated over a time period of 5 hours. The results are shown in Table 3.

TABLE 3
Effect of polymers on biofilm formation

| | | Static biofilm model | |
|---|---|---|---|
| Structure | Strain | % remaining | incubation time |
| 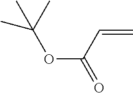 | S. Epidermidis | 100 | 5 h |
| 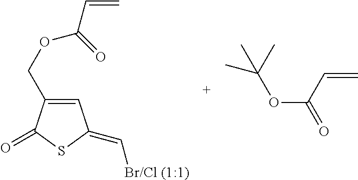 | S. Epidermidis | 100 | 5 h |
| 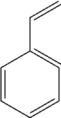 | S. Epidermidis | 100 | 5 h |
| 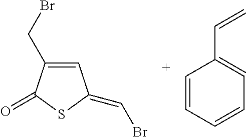 | S. Epidermidis | 33 | 5 h |
| 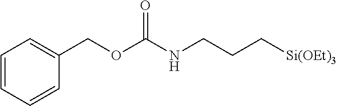 | S. Epidermidis | 100 | 5 h |
| 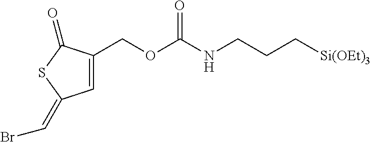 | S. Epidermidis | 45 | 5 h |

### Example 5

Coating and Biological Testing of Steel Samples with Compound F

In the previous Example, Compound F was attached to a glass substrate, where it decreased biofilm formation. The current experiment was performed to determine whether the compound would attach to steel substrates and decrease biofilm formation and thereby biologically induced corrosion in a marine environment.

Coating experiments were performed on stainless steel substrates (30×40 mm²) were received from Ole Øystein Knudsen. To determine a good procedure for coating the substrates, the following samples were prepared and investigated by X-ray photoelectron spectroscopy (XPS):
A) Coated with T_{B}18210, heated to 120°, rinsed, 120° C.
B) Coated with T_{B}18210, rinsed, heated to 120° C., rinsed, 120° C.
C) Rinsed substrate, heated to 120° C., rinsed, 120° C.

Coating Procedure

All substrates were rinsed in an ultrasonic bath for 1 min in dichloromethane, 1 min in acetone and finally 1 min in isopropanol. They were etched for 3 min in 20 wt % HNO₃ at room temperature, then rinsed in RO water and dried at ambient conditions.

A 0.01 M solution of Compound F was prepared by dissolving 0.02366 g in 5 ml toluene.

Two rinsed substrates (for samples A and B) were wiped with the Compound F solution for 1 min A) was immediately put into an oven at 120° C. for 1 hour. B) was first rinsed in toluene to remove any excess T_{B}18210, then placed in an oven at 120° C. for 1 hour. Sample C) was not coated, just heated to 120° C. for 1 hour.

After cooling, all samples were rinsed in toluene and heated at 120° C. for 1 hour.

Results

XPS showed that the Compound F is attached to sample A), but not on sample B). Samples of type B) were therefore not prepared for biological testing.

Samples for Biological Testing

Three samples of each of type A) and C) were prepared for testing of biofilm formation in a marine environment. For comparison, three Cu-covered samples (marked D) were prepared by painting rinsed substrates with “aqua-net” Cu paint from Steen/Hansen Maling. The painted samples were dried at ambient conditions over night.

Biological Testing

Biological testing of coated stainless steel substrates were performed in a static cultures of marine bacteria. The biofilm prevention efficiencies of the coatings were recorded as a) total enumeration of bacteria attached on the steel surfaces and b) most-probable number (MPN) viability testing of attached bacteria. The nature of the thiophenes (expecting to inhibit biofilm generation without being toxic to the bacteria) should account for inhibition of both total counts and viability counts when thiophene-coated surfaces were compared to non-treated controls.

Methods

Three samples of stainless steel substrates were prepared as described above, each of these in triplicate. A primary culture of marine bacteria were prepared by inoculation of 2 ml seawater (collected from 90 m depth through a pipeline system supplying seawater to SINTEF Sealab, Trondheim) in 100 ml Marine Broth 2216 (MB). The cultures was incubated at 20° C. with continuous agitation until significant increase in medium turbidity (cell density appr. 10⁹ cells/ml). Nine 500 ml sterile bottles (PE) with wide necks were prepared with individual steel surfaces placed in 200 ml MB. Primary culture (1.0 ml) was inoculated to each bottle and the cultures with steel surfaces incubated at 20° C. for 14 days with careful continuous agitation, and with medium changes after day 3, 7, and 10. During medium changes 75% of the culture media in each bottle was replaced by fresh MB medium.

At the end of the experimental period (14 days) the steel surfaces were removed from the media and carefully washed in sterile and particle-free (filtered through 0.2 μM sterile filters) seawater. Washed steel surfaces were placed in 200 ml sterile and particle-free seawater and placed in an ultrasound bath for 15 minutes to desorb cells from the surfaces. Desorbed concentrations of bacteria were recorded by epifluorescence microscopy and by MPN-counts in MB medium.

For epifluorescence microscopy 10 ml desorbed samples were stained with 2 μg 3′5-diamino phenylindol (DAPI) and incubated for 5-10 minutes. The stained samples were filtered through 0.2 μm black polycarbonate filters and analysed in a fluorescence microscope at 1250× magnification. For MPN-counts 10-fold serial dilutions of desorbed samples were prepared in sterile seawater and 0.2 ml of each dilution (undiluted to 10⁻⁹ dilution) inoculated in triplicate in 2 ml MB medium in 24-well sterile tissue culture plates. The plates were incubated at 20° C. for 5 days and positive growth recorded as turbidity in the wells. Concentrations of viable bacteria in the desorbed samples were determined from MPN-tables with 95% confidence intervals.

Results

The results from the epifluorescence and MPN-counts are shown in FIG. 3.

FIG. 3 shows total (A) and viable (B) counts of desorbed bacteria from stainless steel surfaces coated with thiophene (Compound F), with no coating and with Cu coating. The results are calculated as concentrations of cells per cm² surface.

The inhibition of bacterial attachment (total counts) and growth (viable counts are shown in FIG. 4.

FIG. 4 shows the inhibition of bacterial attachment (total counts) and growth (median values) for stainless steel surfaces coated with thiophene Compound F and with Cu.

The results of FIGS. 3 and 4 showed that the thiophene inhibited bacterial attachment and viability at a level comparable with Cu, showing appr. 80% inhibition of biofilm attachment and >99% inhibition of viable attached bacteria when median values were compared, although concentrations of viable bacteria differed significantly for the viable counts. While Cu is harmful in high concentrations the thiophene is regarded as a non-toxic chemical. It is important to emphasize that the bacterial concentrations in the surrounding environment was much higher than expected in normal seawater, and that experiments were conducted at temperatures much higher than normal in Norwegian seawaters.

### Example 6

This example relates to the effects of thiophenones according to the invention on quorum-sensing communication between bacteria.

The effect of (Z)-5-(bromomethylene) thiophen-2(5H)-one on AI-1 and AI-2 quorum-sensing by bacteria was tested in a bioluminescence assay as follows:

Inhibition of quorum sensing was assessed as the ability of the furanone to reduce bioluminescence induced by cell free bacterial culture supernatant containing either AI-1 or AI-2 signal molecules. Vibrio harveyi BB886 was used as an AI-1 reporter and Vibrio harveyi BB170 was used as reporter of AI-2 communication.

As a comparison, the corresponding furanone compound was also tested. It was found that the thiophenone compound was considerably more effective in inhibiting quorum-sensing by both AI-1 and AI-2.

The results are set out in FIGS. 5 and 6 for AI-1 and AI-2 quorum-sensing respectively.

1. An agent comprising the compound according to general formula (I): 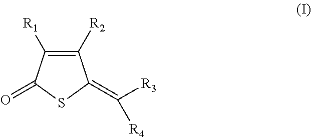
wherein R₁, R₂, R₃ and R₄ are each independently H or a substituent, and wherein at least one of R₁, R₂, R₃ and R₄ is halogen, cyano, cyanate, thiocyanate or C₁-C₆ haloalkyl, with the proviso that the compound is neither nor

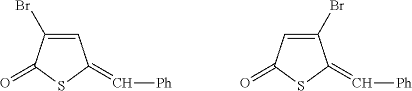

2. An agent for use in medicine, which agent comprises the compound of general formula (II): 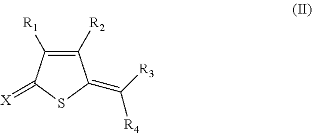
wherein X is O, S, NH or NR′, in which R′ is an optionally substituted C₁-C₆ alkyl group;
R₁, R₂, R₃, and R₄ are each independently H or a substituent, and wherein the compound is capable of blocking or interfering with quorum-sensing microbial communication.

3. An agent for use in medicine, which agent comprises the compound of general formula (II): 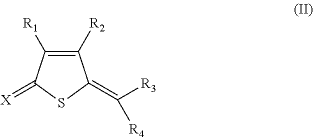
wherein X is O, S, NH or NR′, in which R′ is an optionally substituted C₁-C₆ alkyl group;
R₁, R₂, R₃, and R₄ are each independently H or a substituent, and wherein the compound is capable of preventing or inhibiting biofilm formation.

4. The agent according to claim 2 or claim 3 wherein X is O.

5. An agent according to any preceding claim wherein each substituent is independently a halogen, cyano, cyanate, thiocyanate, alkyl, alkoxy, haloalkyl, alkyl ester, alkylsilyl, alkenyl, alkynyl, aryl, or arylalkyl, which may be substituted or unsubstituted, optionally interrupted by one or more heteroatoms, straight chain or branched chain.

6. An agent according to any preceding claim wherein each substituent is independently halogen, haloalkyl, alkoxy, alkyl ester, phenyl or 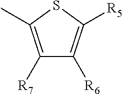
wherein R₅, R₆ and R₇ are each independently H, Br, Cl, OMe, or CHO.

7. An agent according to any preceding claim wherein R₄ is a substituent.

8. An agent according to any preceding claim wherein at least one of R₁, R₃ and R₄ is halogen or C₁-C₆ haloalkyl.

9. An agent according to any preceding claim wherein at least one of R₁ and R₄ is halogen or C₁-C₆ haloalkyl.

10. An agent according to any preceding claim wherein R₁ and R₂ are each independently H or halogen.

11. An agent according to any preceding claim wherein R₃ is H, halogen, C₁-C₆ haloalkyl or phenyl.

12. An agent according to any preceding claim wherein R₄ is halogen, C₁-C₆ haloalkyl or phenyl.

13. An agent according to any preceding claim wherein the halogen is Br.

14. An agent according to any preceding claim wherein R₁ is Br.

15. An agent according to any preceding claim wherein R₄ is Br.

16. An agent according to any preceding claim wherein R₂ is H.

17. An agent according to any preceding claim wherein R₃ is H.

18. An agent according to any of claims 1 to 13 wherein R₁ and R₄ are each Br and R₂ and R₃ are each H.

19. An agent according to any of claims 1 to 13 wherein R₃ and R₄ are each Br and R₁ and R₂ are each H.

20. An agent according to any of claims 1 to 13 wherein R₁, R₃ and R₄ are each Br and R₂ is H.

21. An agent according to any of claims 1 to 13 wherein R₁ is —CH₂—O—CO—(CH₂)₂—COOH and R₄ is Br.

22. An agent according to any of claims 1 to 13 wherein R₁ is thienyl and R₄ is Br.

23. An agent according to any of claims 1 to 13 wherein R₃ is methyl and R₄ is Br.

24. An agent according to claim 5, wherein R₃ is H and R₄ is thiocyanate.

25. A polymer which comprises a compound as defined in any preceding claim.

26. A polymer according to claim 25 which comprises one or more side chain functional groups comprising the compound.

27. A polymer according to claim 25 or claim 26 which comprises a polyacrylate, polymethacrylate, polycrotonate, polyvinyl alcohol, polyvinyl acetate, polystyrene, acrylonitrile or siloxane.

28. A polymer according to any of claims 25 to 27, which is obtainable by polymerising the compound according to general formula (III) with the compound according to general formula (IV):

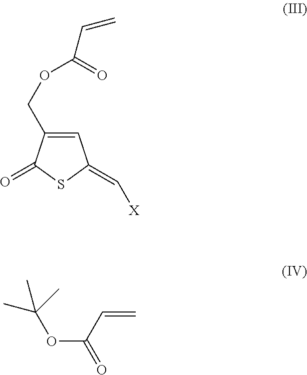

29. A polymer according to any of claims 25 to 27, which is obtainable by polymerising the compound according to general formula (V) with the compound according to general formula (VI):

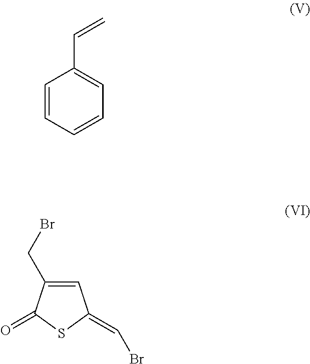

30. An agent or polymer for use in medicine, which comprises the compound as defined in any preceding claim or a pharmaceutically-acceptable salt, ester or prodrug thereof.

31. An agent or polymer according to any preceding claim for use as an antimicrobial.

32. An agent or polymer according to claim 31 for use in the prevention or treatment of microbial infection.

33. An agent or polymer according to claim 32 wherein the microbial infection is a bacterial or fungal infection.

34. An agent or polymer according to any one of claims 31 to 33, which interferes with quorum-sensing microbial communication so as to treat or prevent a condition mediated by microbes which are regulated by quorum-sensing communication.

35. A composition comprising an agent or polymer according to any preceding claim and one or more further antimicrobial agents as a combined preparation for simultaneous, separate or sequential use in the prevention or treatment of microbial infection.

36. A composition according to claim 35 wherein the antimicrobial agent is an antibiotic or an antifungal agent.

37. An agent, polymer or composition according to any of claims 31 to 36 for use in the treatment of periodontitis, gingivitis, dental caries, topical infections, respiratory infections, eye infections, ear infections or localised organ infection.

38. A coating composition comprising the agent or polymer according to any preceding claim.

39. A coating composition according to claim 38 which is capable of binding covalently to a surface.

40. A coating composition according to claim 38 or 39, which comprises the agent is covalently linked to the group Si(OR₅)₃, wherein each R₅ is independently substituted or unsubstituted C₁-C₆ hydrocarbyl.

41. A coating composition according to claim 40, which comprises the compound according to general formula (VII):

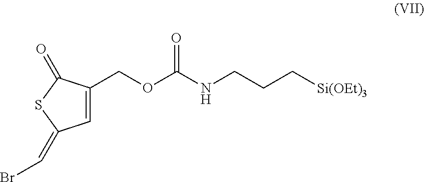

42. An antimicrobial surface cleanser comprising an agent or polymer as defined in any preceding claim.

43. An antimicrobial surface cleanser according to claim 42 which is a disinfectant or cleaning composition.

44. An antimicrobial surface cleanser according to claim 42 or claim 43 wherein the surface is an inanimate surface.

45. An antimicrobial surface cleanser according to claim 42 or claim 44 wherein the surface is the skin of a human or animal.

46. An anti-biofouling composition or coating comprising an agent or polymer as defined in any preceding claim.

47. An anti-biofouling composition according to claim 46 wherein the biofouling occurs on marine vessels, sub marine installations, pipelines, water pipes, industrial machines or installations, water systems, cooling towers, heat exchangers, chain lubrication systems, oil or gas platforms, fish-farming installations or surfaces, machines, tools or devices used in food production.

48. A personal care product comprising an agent or polymer as defined in any preceding claim which is a personal hygiene article, shampoo, soap, deodorant, dentifrice, mouthwash, contact lens solution or cosmetic composition.

49. A medical device or implant which is coated with the coating composition as defined in any of claims 38 to 41.

50. A medical device or implant according to claim 49, which is a catheter, artificial heart valve, surgical pin, pacemaker capsule, prosthetic joint, stent, shunt, endotracheal or gastrointestinal tube, surgical or dental instrument, surgical suture, dental implant, electrode, dialysis device or bandage.
